(19) United States
(12) Patent Application Publication
(10) Pub. No.: US 2007/0031398 A1
(43) Pub. Date: Feb. 8, 2007

(54) One of several enzyme-containing compounds, dietetic food products and drugs produced from and the use therefor for medical purpose

(75) Inventor: Winfried Miller, Kempten (DE)

Correspondence Address: JOYCE VON NATZMER;Hall, Vande Sande & Pequigot, LLP, 10220 River Road, Suite 200, Potomac, MD, 20854, US

(73) Assignee: MUCOS PHARMA GmbH & Co., Geretsried (DE)

(21) Appl. No.: 10/555,244

(22) PCT Filed: May 14, 2004
(86) PCT No.: PCT/EP04/05213

§ 371 Date: Oct. 31, 2005

(30) Foreign Application Priority Data
May 14, 2003 (DE) 103 21 725.8

(51) Int. Cl. A61K 38/48; A61K 31/715; A61K 31/353; A61K 31/12; A61K 31/015

(52) U.S. Cl. 424/94.63; 514/54; 514/27; 514/456; 514/690; 514/763

(57) ABSTRACT

The invention relates to one or several enzyme-containing compounds, i.e. to one or several hydrolyses and to one or several antioxidants which are selected from ubiquinones, carotinoides and antioxidative vitamins. The inventive compounds are used in the form of components for producing food products or drugs which are used, inter alia as dietetic food supplements, in particular for a supplementary dietetic regime and for treating immune system disorders.

The present invention relates to the area of enzyme-containing compositions and of food products, in particular dietetic food supplements, which are used inter alia as supplementary balanced diet, and of medicaments which comprise these enzyme-containing compositions or consist of these enzyme-containing compositions. It relates more exactly to enzyme-containing compositions, food products and medicaments which comprise hydrolases and further medicinally active ingredients, and to their use inter alia for all illnesses resulting from a dysregulation of the immune system, both through internal and external influences.

For the purposes of the present invention, balanced and supplementary balanced diet mean a food product which is taken by patients under medical supervision for particular medical purposes for the treatment of illnesses.

Food supplements are food products which are supplied in the form of tablets, capsules or as powder. They serve to supplement the diet with certain substances such as, for example, vitamins, minerals, trace elements and bulking agents. For this purpose, these substances are ordinarily present in an increased concentration. Dietetic food supplements mean products which themselves have only a small average calorific value and nutritional value and therefore can also be consumed as part of diets.

Food supplements assist in improving the nutrition of healthy people who, for example because of their normal routine, do not have the opportunity or find the time to routinely consume all the required nutrients to a sufficient extent with the normal diet.

However, food supplements can also be used to compensate deficiency states caused by an illness or the treatment of an illness. The targeted compensation of a deficiency of essential micronutrients and/or food constituents that have a protective effect with (e.g. dietetic) food supplements, which is often not possible with the normal diet in various illnesses, can improve the result of therapy and the patient's quality of life. Food supplements are thus able in general to improve the state of health of the users and in people with illnesses to have a beneficial effect in particular on the progress of the disease and the result of treatment.

Food supplements are generally not medicaments. However, they may, depending on the area of use, the chosen ingredients and the dosage thereof, also be medicaments.

The invention relates to specific compositions which comprise active ingredients intended for food supplementation, and to the food products and medicaments obtainable therefrom, which may additionally comprise excipients, for example for pharmaceutical formulation. With these compositions, food products or medicaments, the supplying of essential nutrients to healthy people and people with illnesses is improved, while in addition in the case of people with illnesses the result of a therapeutic treatment is enhanced.

Enzyme-containing products are known in the art. WOBE-Mugos® comprises trypsin, chymotrypsin, papain and thymus extract. It is used inter alia for supportive long-term treatment of malignant tumors. Wobenzym® comprises a mixture of animal pancreatic enzymes and plant enzymes from Ananas comosus and Caica papaya, plus rutoside. It is used for supportive treatment of inflammations and vascular disorders.

Starting from this prior art, it is an object of the present invention to indicate compositions, food products and medicaments with modified chemical composition, which have an improved efficacy as health-promoting food products, for example in the form of food supplements, and as medicaments in the medical sector and which are suitable for novel uses and indications.

In a first aspect, the invention relates to compositions which comprise one or more hydrolases and one or more antioxidants which are selected from vitamins having antioxidant activity, carotenoids, selenium-containing substances, ubiquinones.

Hydrolases are enzymes which hydrolytically cleave ester, ether, peptide, glycoside linkages, etc. They include esterases, phosphatases, glycosidases and proteases which can be used according to the invention. Besides the naturally occurring hydrolases obtained from natural products, it is also possible to use hydrolases which are produced artificially, e.g. by genetic manipulation. Hydrolases can be obtained industrially from plants, animal organs, bacteria (proteases for example from Brevibacterium linens) and fungi. Bacteria or other microorganisms are used for obtaining them by genetic manipulation. The hydrolases can be used singly or in a combination of a plurality of hydrolases. Examples of hydrolases which can be used according to the invention are indicated hereinafter.

Antioxidants prevent or delay even in lower concentration the oxidative decomposition of active substances. In the human body they protect from cellular damage and have an anticarcinogenic effect. The antioxidants can be used singly or in a combination of a plurality of antioxidants. Examples of antioxidants from the classes of vitamins having antioxidant activity, carotenoids, selenium-containing substances, ubiquinones which can be used according to the invention are indicated hereinafter.

Combination of one or more hydrolases with one or more antioxidants results in compositions which have an increased health-promoting effect going beyond the effect of the individual substances, both for healthy people and for people with illnesses, especially for people suffering from dysregulation of the immune system. Included herein are chronic or acute immuno-deficiency, an impairment of the body's defenses, inflammatory-rheumatic disorders and all other inflammatory, immunological and neoplastic disorders. For the treatment, the composition can be employed in the form of a dietetic food product for a supplementary balanced diet. The improved nutrition enhances the health of people with illnesses. The people with illnesses may be patients with rheumatism or people with immunodeficiency and, in particular, tumor patients, who are undergoing a therapy serving to control the tumor. This therapy may comprise a surgical procedure, a chemotherapy and/or a radiotherapy, during the course of which the patients have an increased need for essential micronutrients and food constituents having a protective effect.

Essential micronutrients mean in this context the antioxidants used according to the invention, such as vitamins, carotenoids, selenium-containing substances, ubiquinones, as long as they have appropriate antioxidant effects. Food constituents having a protective effect mean inter alia the hydrolases used according to the invention, such as plant and animal proteases. The increased requirement for these substances is covered by the compositions, food products and medicaments of the invention.

The above compositions of the invention may additionally comprise one or more polyphenols, one or more flavonoids and/or one or more flavonoid-containing substances.

The compositions of the invention listed above may additionally comprise one or more amino acids and/or one or more polysaccharides.

In a second aspect, the invention relates to compositions which comprise one or more hydrolases and one or more amino acids. These compositions also have advantageous immunomodulatory properties.

In a third aspect, the invention relates to compositions which comprise one or more hydrolases and one or more polysaccharides, which just like the compositions of hydrolases and amine acids can be administered inter alia for improving the state of health, in particular improving the immune status, in the form of food products or medicaments. In both cases it has surprisingly been found that these advantageous properties can be achieved even without the presence of antioxidants.

The invention further relates to the provision of compositions which comprise hydrolases in combination with amino acids and polysaccharides.

In a fourth aspect, the invention also relates to the compositions which comprise exclusively hydrolases as active constituents and are combined where appropriate with excipients which are pharmaceutically acceptable or approved in food products.

The invention accordingly relates to the compositions (I) to (X) which comprise the following active constituents or consist thereof:
(I) hydrolases+antioxidants, namely vitamins and/or carotenoids and/or selenium-containing substances and/or ubiquinones;
(II) composition (I)+polyphenols and/or flavonoids and/or flavonoid-containing substances;
(III) composition (I) or (II)+amino acids and/or polysaccharides;
(IV) hydrolases+amino acids;
(V) hydrolases+polysaccharides;
(VI) composition (IV)+polysaccharides;
(VII) composition (IV)+polyphenols and/or flavonoids and/or flavonoid-containing substances;
(VIII) composition (V)+polyphenols and/or flavonoids and/or flavonoid-containing substances;
(IX) composition (VI)+polyphenols and/or flavonoids and/or flavonoid-containing substances;
(X) hydrolases,
   where the above statements such as, for example, “amino acids” are to be understood as statements of classes of substances from which one or more representatives may be present in the compositions of the invention. “Amino acids” accordingly means “one or more amino acids”, for example “L-arginine” or “L-arginine+L-cysteine”, etc., and the same applies to all other stated classes of substances.

The hydrolases are preferably plant proteases and/or animal proteases which are obtained from natural products, represent the natural products themselves or are produced by synthesis or genetic manipulation.

The plant proteases preferably comprise pure bromelain, pineapple extracts, pure papain and papaya extracts as they are used singly or in combination. Bromelain consists of a plurality of enzymes having proteolytic activity, more accurately endopeptidases, which occur both in ripe and unripe pineapple (Ananas comosus) and are obtained from the stalks of the seed heads.

Bromelain has inter alia an anti-inflammatory effect. Papain is a proteinase which is obtained from the latex of unripe papaya fruits. Papain and bromelain can particularly preferably be used in combination. The enzymes can be used in pure form or in the form of plant extracts as long as they comprise the plant proteases, inter alia in the form of pineapple extracts and papaya extracts. It is likewise preferred to use ficin obtained from figs, or fig extracts, which is as papain and bromelain, a cysteine proteinase.

The animal proteinases used according to the invention preferably comprise trypsin and/or chymotrypsin and/or pepsin (peptidyl-peptide hydrolase). Trypsin is obtained for example from the acidic pancreatic juice from pigs by fractional precipitation in aqueous medium and by activating trypsinogen in the slightly alkaline pH range. This results in a mixture of α-, β- and γ-trypsin, which is able to hydrolyze peptide, amide and ester linkages if the basic amino acids arginine and lysine are involved in these linkages. Chymotrypsin is prepared from the acidic pancreatic juice of cattle by extraction in aqueous medium and subsequent fractional precipitation. Chymotrypsin is converted into the active form in a slightly alkaline medium. Chymotrypsin is obtained in crystalline form by further technical processing steps (ultrafiltration, chromatographic purification) and can be used in this form. The obtained mixture of chymotrypsin A and B hydrolyzes peptide, amide and ester linkages if the aromatic amino acids tyrosine, phenylalanine and tryptophan, and leucine, methionine and glutamic acid are involved in these linkages. As proteolytic enzyme, pepsin also cleaves circulating immunoglobulins of the IgG type, so that the IgG molecule is decomposed into an Fc fragment and a bivalent F(ab)2 fragment.

Bromelain has a potent antiodematous effect and can thereby eliminate swellings more quickly than other proteases. Trypsin likewise has antiodematous activity. Trypsin and chymotrypsin have a major effect on blood coagulation and thus on the flow properties of blood. With the combination of papain and trypsin it is possible to achieve a particularly strong C1Q binding reduction (complement region of the immune system). The two proteases alter this binding region in such a way that no binding can take place. Inflammatory reactions are thereby inhibited.

Bromelain and papain are potent cleavers of immune complex compounds which are regarded as markers of a dysregulated immune system. High immune complex concentrations are repeatedly measured in particular in association with autoimmune diseases and oncological disorders (cancer). Elimination thereof is the precondition for unimpaired microphage activity.

All proteases act on the expression of so-called cell surface adhesion molecules. Communication between cells takes place inter alia via these adhesion molecules. Modulation of the adhesion molecules is distinctly improved by the combination of proteases.

Administration of single enzyme products (e.g. bromelain alone) has by far not the therapeutic effect as administration of enzyme combinations. With the combination of bromelain and trypsin it was possible to find a superadditive effect with respect to the reduction of the inflammatory event. This is shown by a distinctly shorter healing phase upon use of enzyme combinations than in the case of monoenzyme products.

The combination of plant and animal proteases activates resting monocytes and NK cells, thus stimulating the immune system. On the other hand, the enzyme combinations of the invention downregulate overstimulated, overactivated immune cells.

These effects are particularly pronounced when the proteases are combined with antioxidants, especially selenium-containing substances and in particular sodium selenite, flavonoid-containing substances, etc., amino acids and optionally polysaccharides.

Examples of compositions (X) preferred according to the invention are compositions which comprise bromelain and/or papain and/or trypsin and/or chymotrypsin, without other active substances, optionally combined with excipients which are pharmaceutically acceptable or approved in food products such as bromelain+papain+trypsin+chymotrypsin.

Examples of other hydrolases which can be used according to the invention are: α-amylase, β-amylase, α-D-galactosidase, glucoamylase, lipases, proteinases, tannases, invertase, lysozyme, pullulanase, thio-glucosidase, lactase, pectinolases, α-L-rhamnosidase, which can be employed singly or in the form of mixtures, also with the above plant and animal proteases.

The hydrolases can be employed over a wide concentration range. The lower limit corresponds to the concentration below which the desired effect on health or therapeutic effect is no longer present. The upper limit can be defined as the concentration above which there is no further increase in activity and thus only the costs of manufacturing the product rise.

The hydrolases such as bromelain and papain are preferably used in a total concentration of from 20 to 60% by weight, even better 30 to 50% by weight, based on the total amount of active constituents in the composition. If the composition comprises two or more than two hydrolases, these are advantageously used in similarly large amounts, e.g. 25% by weight (or, 15% by weight) papain and 25% by weight (or 15% by weight) bromelain. However, it is also possible for the hydrolases to be used in different concentrations, such as 15% by weight animal protease(s) and 35% by weight plant protease(s) and vice versa, or 15% by weight of a first plant protease such as bromelain, and 35% by weight of a second plant protease such as papain, or vice versa.

In a preferred embodiment of the invention, the antioxidants comprise vitamins which are selected from vitamin A, C and E and the esters of vitamin A and E which can be used in food products and medicaments. Examples of such esters are the acetates, the aspartates and the tartrates.

It is possible to use supportively vitamin K, vitamin B1 (thiamine), B2 (riboflavin), B6 (pyridoxine), B12 (cyano-cobalamin), niacin, panthotenic acid, biotin, folic acid, myo-inositol, which themselves have no antioxidant effect, however. The vitamins of the B group all have a coenzyme function, so that oral administration thereof, especially in combination with amino acids and/or polysaccharides, is physiologically advantageous.

The vitamins are used singly or in a mixture in the composition of the invention. Their total concentration is for example in the range from 30 to 60% by weight, even better 40 to 50% by weight, based on the active constituents of the composition if no other antioxidants are used. If other antioxidants are present, the vitamins having antioxidant activity can be employed in lower concentration.

An antioxidant effect is also shown by carotenoids, which are secondary plant constituents, such as lycopene, a yellowish red plant pigment with a polyene structure, which occurs in rosehip, tomatoes and other fruits. Lycopene is a polyunsaturated polyene and has proved to be a very effective natural free-radical scavenger. It can therefore be employed particularly advantageously in combination with the abovementioned antioxidant vitamins. The combination with lycopene results in a surprisingly increased efficacy in the therapy of rheumatism and in concomitant treatment within the framework of a cancer therapy. The proportionate amount of the carotenoid, such as lycopene, as constituent of the total amount of antioxidants is, for example, from 5 to 20% by weight based on the total amount of the active constituents, even better from 9 to 16% by weight.

Further carotenoids which, like lycopene, can be employed as antioxidants in the compositions of the invention are α-, β-, γ-carotene, neurosporin, phytofluene, phytoene from the group of carotenes and xanthophyll, violaxanthin, crocetin, astaxanthin, capsanthin from the group of xanthophylls.

The compositions of the invention may additionally comprise one or more flavonoids which are incorporated in the form of pure flavonoids or in the form of natural products comprising flavonoids. Flavonoids belong to the class of polyphenols or plant phenols (PHP=polyhydroxyphenols).

The flavonoids can be selected according to the invention from flavones, flavonols, flavanols, isoflavanols. These include besides quercetin, quercetin glycosides, such as rutin, the flavanones eriodictyol, hesperetin, liquiritigenin, naringenin, pinocembrin, the flavanolol taxifolin, the flavones apigenin, chrysin, diosmetin, eupatorin, luteolin, scutellarein, scutellarein tetramethyl ether, sinensetin and the flavonols axillarin, chrysoplenetin, chrysoplenol, eupaletin, eupatoletin, fisetin, galangin, gossypetin, isorhamnetin, jaceidin, kaempferol, myricetin, patuletin, quercetagenin, rhamnetin, robinetin, spinacetin.

Flavonoids which are particularly preferred according to the invention are: quercetin, hesperetin, pinocembrin, taxifolin, chrysin, luteolin, kaempferol, rhamnetin.

Suitable flavonoid-containing substance(s) are onion powder, buckwheat extracts, grapeseed flavonoids and citrus flavonoids. The grapeseed and citrus flavonoids comprise oligomeric procyanidins (OPC) which belong to the group of flavonols. Standardized OPC comprises at least 50% or more pine bark OPC (Pinus maritima) and grapeseed OPC (Vitis vinifera). Grapeseed OPC comprises for example up to 20% catechin, epicatechin and epigallocatechin, pine OPC up to 10% catechin and up to 10% taxifolin. These oligomeric procyanidins can likewise be used because of their antioxidant effect in the compositions of the invention.

Particular preference is given to quercetin and the glycosides of quercetin such as, for example, rutin (rutoside). Rutoside is normally used as pain remedy but also, like the other flavonoids too, shows surprisingly, good properties in the therapies of the invention, i.e. in the use as balanced micronutrient in dietetic food products for strengthening the immune system, for strengthening the body's defenses, for treating inflammatory rheumatic disorders and for regulating the immune system in situations of physical and/or mental stress.

The flavonoids and the flavonoid-containing natural products can be used singly or as mixture of two or more of these substances. They are advantageously used in a concentration of from 10 to 50% by weight based on the total amount of active constituents. Higher and lower concentrations are, however, equally conceivable. The daily dose on use of grapeseed flavonoids and pine bark can advantageously be 150 mg.

Very good results are achieved in the concomitant treatment of cancer and rheumatism, but also in the strengthening of the body's immune defenses, if the compositions of the invention additionally comprise one or more selenium-containing substances having antioxidant effects, such as selenium yeast and/or alkali metal selenites such as sodium selenite, alkaline earth metal selenites such as magnesium selenite, ammonium selenite, the corresponding selenates or selenamino acids (Se-methionine, Se-cystine). Selenium is an essential trace element. It occurs naturally as a constituent of glutathione peroxidase, of thioredoxin reductase and of thyroxine 5-deiodase. Selenium acts as intracellular antioxidant. It acts as free-radical scavenger to counter cell damage due to free-radical formers or lipid hydro-peroxides. Selenium modulates for example the function of lymphocytes and can increase the activity of natural killer cells. Selenium has an effect on the DNA repair mechanism and may be involved in the initiation of apoptosis. In the compositions of the invention it leads to a synergistic increase in the immunomodulatory properties of the compositions. Selenium in its above forms is, as an essential trace element with antioxidant effect, an ideal combination partner according to the invention with the antioxidants vitamin E and vitamin C.

Sodium selenite is particularly preferred. Sodium selenite (as well as other alkali metal and alkaline earth metal selenites), owing to its chemical structure, is absorbed very well after administration, in particular oral administration, of a composition of the invention, and it displays the desired catalytic activity directly in the target cells. Sodium selenite accordingly has particularly good bioavailability and particularly high biological activity. This is advantageous for example when a sodium selenite-containing composition of the invention is used in order to protect, without a time lag and highly effectively, the healthy body cells of a cancer patient from the cell-damaging effects of chemotherapy/radiotherapy when the chemotherapy or radiotherapy is carried out. The rapid bioavailability and the high biological activity of the sodium selenite ensure that intake of the composition of the invention on the day of the cancer therapy reliably leads within a short time to the desired cytoprotection. The selenomethionine present in selenium yeast must by contrast be first converted into the inorganic precursor before it can become active in the available selenium pool. The abovementioned effects thus occur with a time-lag in the case of selenium yeast. In addition, there is no risk during long-term administration of sodium selenite that selenium accumulates in a form which is inactive and, as the case may be, even harmful to health.

After oral administration of sodium selenite to subjects it is possible to measure a significant reduction in interleukin 4 (IL-4) and a significant increase in the cytokine IFN-γ in the serum. It is additionally possible after oral administration of sodium selenite to find marked changes in the immune status: the CD8+ T cells decrease significantly. The disturbance of the immune balance which is always found in chronic illnesses can be modulated by sodium selenite in the direction of immunohomeostasis. A demonstrable equilibrium between T-helper cells of type 1 (Th1), T-helper cells of type 2 (Th2), cytotoxic T cells and CD8+ cytotoxic T cells results.

Hydrolases, more accurately proteases, especially combinations of proteases such as bromelain+papain or bromelain+papain+trypsin and/or chymotrypsin, improve through their immunomodulatory effect the immunomodulatory properties of sodium selenite. The one-sided high stimulation of the immune system with, resulting therefrom, an imbalance of the various immune system parameters can be prevented by concurrent administration of sodium selenite together with proteases. This positive synergistic effect can be exploited for all chronic illnesses and neoplastic disorders.

The selenites can be present in a concentration of from 0.001 to 0.3% by weight, better 0.01 to 0.1% by weight, based on the total amount of active constituents. A daily therapeutic dose of from 50 to 300 μg has proved to be particularly good.

A further group of substances which is used according to the invention in combination with the hydrolases or as preferred addition in the other compositions of the invention is the group of amino acids. Glycine, L-alanine, L-serine, L-threonine, L-valine, L-leucine, L-isoleucine, L-aspartic acid, L-asparagine, L-glutamic acid, L-glutamine, L-arginine, L-lysine, L-hydroxylysine, L-ornithine, L-citrulline, L-cysteinee, L-cystine, L-methionine, L-tyrosine, L-phenylalanine, L-tryptophan, L-histidine, L-proline, L-hydroxyproline can be used singly or in combination, as can their esters and salts which are acceptable pharmaceutically and for food products, as long as they form esters and salts.

Glycine, L-arginine, L-glutamine and L-methionine have proven to be particularly advantageous. L-arginine is most preferred.

Amino acids likewise have a role in modulating the immune system. They influence signal transmission (signal transduction) of the immune system via cell surface and cytokine modulation and via influencing the redox potential of the system. Arginine is involved in the synthesis of polyamines. These in turn are crucially involved in cell division, DNA synthesis and regulation of the cell cycle. Arginine also has an effect via these mechanisms on the proliferation of lymphocytes, on the cytolytic capacity of macrophages and NK cells and on the cytokine balance (IL1). Arginine is the principle endogenous initial metabolite for nitric oxide (NO) and additionally has a strong influence on the hemodynamic situation. Overexpression of the cytokine TGF-β occurs in acute and chronic illnesses, including cancers. The amino acid arginine locally reduces the overexpression of the cytokine TGF-β, at the site of the inflammation. This effect is likewise ensured by the combination according to the invention with proteases.

The concentration of the amino acids is advantageously in the range from 5 to 15% by weight, even better 7 to 12% by weight, based on the total amount of active constituents.

Owing to their good immunomodulatory properties, the compositions of the invention additionally comprise hydrolases and polysaccharides or preferably comprise such polysaccharides selected from amylose, pustulan, dextran, amylopectin, cellulose, arabans, arabinogalactan, fructans, glucans such as β-glucan, mannans, glycogen, chitin, pectins, locust bean gum, tamarind gum, guar, singly or in a mixture, in combination with antioxidants, wherein β-glucan has particularly advantageous properties.

They are preferably used in a concentration in the range from 5 to 10% by weight based on the total amount of active constituents.

β-Glucan stimulates macrophage activity and leads to an increase in the number of NK cells and of T lymphocytes. As immunomodulating substance, it thus represents an optimal combination partner for proteases, especially in compositions (III), (V), (VI), (VIII) and (IX) indicated above.

To improve the function of cells belonging to the immune system, the compositions of the invention can be additionally mixed with minerals such as potassium, calcium, magnesium, iron, zinc, manganese or germanium, each in the form of inorganic salts, salts of organic acids or organic compounds which comprise these metals in complexed form. Zinc and manganese compounds act as coenzymes for the proteases and thus assist the biochemical and physiological processes of cells. Zinc is an essential trace element for protein and nucleic acid metabolism. It converts vitamin A in the cell into the active form. Zinc shows a marked synergistic effect with vitamin C. Germanium has a central role in the immune system, stimulates interferon synthesis and is important in all immunologically triggered illnesses. Germanium is therefore important in all neoplastic disorders and rheumatic disorders.

The following active substances are particularly preferred from the medical viewpoint according to the invention because of their advantageous range of effects:

from the class of substances

hydrolases: bromelain, papain, trypsin, chymotrypsin, singly or as combination of two to four hydrolases,
vitamins: vitamin A, E and C, singly or as mixture of these vitamins,
carotenoids: lycopene, α-, β-, γ-caroene, xantophyll, singly or as mixture of these carotenoids,
selenium-containing substances: sodium selenite,
flavonoids (polyphenols—PHP): hesperetin, naringenin, pinocembrin, taxifolin, chrysin, eupatorin, luteolin, chrysoplenetin, chrysoplenol, eupaletin, galangin, gossypetin, kaemperol, quercetin, singly or in the form of a mixture,
amino acids: L-arginine, L-methionine, L-glycine, L-glutamine, singly or in combination,
polysaccharides: arabans, fructans, glucans, mannans, amylopectin, singly or in a mixture of a plurality of polysaccharides,
minerals/trace elements: potassium, calcium, magnesium, iron, zinc, manganese or germanium, singly or in combination.

The effect of these combinations of proteases, vitamins, amino acids and trace elements on the immune system is stronger than is to be expected from the individual substances. The immune system homeostasis as precondition for a balanced immune system can be restored with the aid of the compositions of the invention after the immune system has responded to acute or chronic disturbing factors distinctly more quickly when the actuators of the immune system, the cytokines and the cell surface adhesion molecules, are modulated and influenced from all sides. This is efficiently achieved within an acceptable time only by the above combination of various immunomodulatory substances.

Compositions of the invention having particularly advantageous properties are indicated below. They have the following qualitative composition in terms of the active constituents:
papain, bromelain, lycopene, vitamin E acetate, vitamin A acetate and sodium selenite (cf. example 1);
papain, bromelain, lycopene, vitamin E acetate, β-carotene, grapeseed flavonoids and sodium selenite (cf. example 2);
papain, bromelain, β-carotene, citrus flavonoids, L-arginine and β-glucan (cf. example 3).
papain, bromelain, vitamin E acetate, rutin-containing buckwheat extract, quercetin-containing onion powder and L-arginine (cf. example 4).

These compositions of the invention may additionally comprise one or more animal proteases, e.g.: trypsin or chymotrypsin or pepsin; trypsin+chymotrypsin; trypsin+chymotrypsin+pepsin; chymotrypsin+pepsin; trypsin+pepsin.

The quantitative data follow in examples 1 to 4 which relate to the same compositions. The amounts in which these active constituents are present in the compositions of the invention are, as mentioned hereinbefore for the hydrolases, not critical. The lower limit is at the amounts below which the desired effect is no longer detected. The upper limit is to be regarded as the amount of the respective active constituent above which no further increase in the effect is observed.

The compositions of the invention can be prepared by simply mixing the active constituents and are then present in the form of a powder. They may comprise exclusively the indicated active substances or comprise the latter in addition to conventional excipients. However, to prepare particular dosage forms such as tablets, film-coated tablets with enteric coating, capsules, sugar-coated tablets, effervescent tablets, suppositories, enemas, they are mixed with customary pharmaceutical excipients. Among those used for this purpose are: microcrystalline cellulose, talc, colloidal silicon dioxide, stearic acid, magnesium stearate, magnesium oxide, fish gelatin, crosspovidone, calcium silicates, hydroxypropylcellulose, hydroxymethylcellulose and titanium oxide. The pharmaceutical preparation of the various dosage forms is not critical and is known in the art.

The present invention further relates to food products, in particular for a supplementary balanced diet, for strengthening the immune defenses or the body's defenses, for treating inflammatory and inflammatory-rheumatic disorders, for regulating the immune system in situations of physical and/or mental stress, for strengthening the immune system before, during and/or after an oncolytic treatment or for reducing the risk of tumor recurrences and secondaries which comprise one of the compositions of the invention described hereinbefore or, if no additives are used, comprise such a composition.

The present invention additionally relates to medicaments which comprise a composition of the invention described hereinbefore or consist thereof, for strengthening the immune defenses or the body's defenses, for treating inflammatory and inflammatory-rheumatic disorders, for regulating the immune system in situations of physical and/or mental stress, for strengthening the immune system before, during and/or after an oncolytic treatment or for reducing the risk of tumor recurrences and secondaries.

The invention additionally relates to the use of the compositions of the invention for dietetic treatment, in particular for a supplementary balanced diet, for strengthening the immune defenses or the body's defenses, for treating inflammatory and inflammatory-rheumatic disorders, for regulating the immune system in situations of physical and/or mental stress, for strengthening the immune system before, during and/or after an oncolytic treatment or for reducing the risk of tumor recurrences and secondaries.

An example which is indicated of the above uses is the concomitant therapy during treatment of carcinomas in which the composition indicated in example 1 can be used. A healthy and complete diet is ensured with the composition and the food products produced therefrom.

This applies both to the period before, during and at defined intervals after the tumor-destructive therapy, e.g. an operation, chemotherapy and/or radiotherapy. The reduced intake of micronutrients (e.g. vitamins, trace elements) which is caused by nausea, reduced food intake, vomiting, diarrhea and sweating is compensated. This improves the patient's tolerance of the treatment, increases the response to the therapy and improves the patient's quality of life. The proteases reduce the side effects of the therapy, the lycopene shows prophylactic and therapeutic effects. For example, it was possible to find a reduction in the incidence and aggressiveness of prostate carcinomas. Overall, the efficacy of the tumor-destructive therapy is improved.

Administration of the defined balanced micronutrients such as vitamin A, vitamin E, sodium selenite, lycopene, bromelain and papain can be divided into two sections: a first section which represents the active treatment phase with an increased dose of the composition, which is followed by a second section in which a maintenance dose is administered over the course of 4-weeks. This dose normally comprises 50% of the dose of the active treatment phase.

Finally, the present invention relates to the use of the compositions of the invention described hereinbefore for producing the medicaments for the indications indicated above as well as others.

Four examples of compositions of the invention and their indications are indicated below.

Besides the active constituents, the compositions comprise conventional additives or excipients for formulating food products or medicaments, namely microcrystalline cellulose, talc, colloidal silicon dioxide, stearic acid. The quantitative data are data in milligrams based on a single dose to be administered of the respective composition.

### EXAMPLE 1

### Composition for Dietary Supplementation in Association with a Tumor-Destructive Treatment

| | |
|---|---|
| Papain | 50 |
| Bromelain | 50 |
| Lycopene | 25 |
| Vitamin E acetate | 72 |
| Vitamin A acetate | 3 |
| Sodium selenite | 0.042 |
| Microcrystalline cellulose | 186 |
| Talc | 10.9 |
| Colloidal silicon dioxide | 3 |
| Stearic acid | 10 |

The composition is employed for supportive therapeutic treatment in association with tumor-destructive treatments. The above composition is administered orally in the form of tablets a maximum of four times a day. Administration takes place in the form of a powder, sugar-coated tablet, tablet or film-coated tablet with enteric coating.

### EXAMPLE 2

### Composition for Treating Inflammatory-Rheumatic Disorders

| | |
|---|---|
| Papain | 33 |
| Bromelain | 34 |
| Lycopene | 16 |
| Vitamin E acetate | 48 |
| β-Carotene | 5 |
| Grapeseed bioflavonoid | 33 |
| Sodium selenite | 0.028 |
| Microcrystalline cellulose | 207 |
| Talc | 11.0 |
| Colloidal silicon dioxide | 3.0 |
| Stearic acid | 10 |

The composition is employed in the form of a food product as powder, sugar-coated tablet, tablet or film-coated tablet with enteric coating for a supplementary balanced diet for the treatment of rheumatoid disorders. It is administered orally up to four times a day.

### EXAMPLE 3

### Composition for Strengthening the Immune-System

| | |
|---|---|
| Papain | 33 |
| Bromelain | 34 |
| β-Carotene | 5 |
| Citrus bioflavonoid (45%) | 33 |
| L-arginine HCl | 16 |
| β-Glucan | 9.0 |
| Microcrystalline cellulose | 207 |
| Talc | 11.0 |
| Colloidal silicon dioxide | 3.0 |
| Stearic acid | 10 |

The composition is employed in the form of a food product as powder, sugar-coated tablet, tablet or film-coated tablet with enteric coating for a supplementary balanced diet for strengthening the immune system. It is given in from one to four doses per day, each of which corresponds to the above dosage.

### EXAMPLE 4

### Composition for a Supplementary Balanced Diet for Strengthening the Immune System, for Strengthening the Body's Defenses and for Dietetic Treatment of Inflammatory-Rheumatic Disorders

| | |
|---|---|
| Papain (papaya extract) | 50 |
| Bromelain (pineapple extract) | 50 |
| Vitamin E acetate | 50 |
| Buckwheat extract (rutin) | 100 |
| Onion powder (quercetin) | 50 |
| L-arginine HCl | 25 |
| Magnesium oxide | 50 |

The composition is used in the form of a powder, sugar-coated tablet, a tablet or film-coated tablet with enteric coating as food product for strengthening the immune defenses. It is administered orally a maximum of four times a day.

1. A composition comprising:
one or more hydrolases, and
(a) one or more antioxidants which are selected from the group of vitamins having antioxidant activity, carotenoids, selenium-containing substances, and ubiquinones,
(b) one or more amino acids,
(c) one or more polysaccharides, or
(d) combinations thereof.

2. The composition of claim 1 comprising one or more hydrolases and one or more amino acids.

3. The composition of claim 1 comprising one or more hydrolases and one or more polysaccharides.

4. The composition of claim 1 comprising one or more hydrolases, one or more amino acids and one or more polysaccharides.

5. The composition of claim 1, further comprising one or more polyphenols, one or more flavonoids and/or one or more flavonoid-containing substances.

6. The composition of claim 5 comprising one or more amino acids and/or one or more polysaccharides.

7. The composition of claim 1, wherein said composition comprises one or more hydrolases and one or more amino acids; one or more hydrolases and one or more polysaccharides; or one or more hydrolases, one or more amino acids and one or more polysaccharides, further comprising one or more flavonoids and/or one or more flavonoid-containing substances.

8. The composition of claim 1, wherein the hydrolase(s) are plant proteases and/or animal proteases.

9. The composition of claim 8, wherein the plant protease(s) is/are bromelain, bromelain-containing pineapple extracts, papain, papain-containing papaya extracts or combinations thereof, and the animal protease(s) is/are trypsin or chymotrypsin or combinations thereof.

10. The composition of claim 1, wherein the vitamin(s) having antioxidant activity is/are selected from vitamin A, C and E or the esters of vitamin A and E.

11. The composition of claim 1, wherein the selenium-containing substance(s) having antioxidant activity is/are selenium yeast, alkali metal selenites such as sodium selenite, alkaline earth metal selenites such as magnesium selenite, or ammonium selenite, or the corresponding selenates.

12. The composition of claim 5, wherein the flavonoid(s) is/are flavones, flavonols, flavanols, or isoflavanols.

13. The composition of claim 5, wherein the flavonoids comprise quercetin, rutin, hesperetin, pinocembrin, taxifolin, chrysin, luteolin, kaemperol, and/or rhamnetin.

14. The composition of claim 5, wherein the flavonoid-containing substance(s) is/are onion powder, buckwheat extracts, grapeseed flavonoids or citrus flavonoids.

15. The composition of claim 1, wherein said composition comprises the ubiquinone is coenzyme Q10.

16. The composition of claim 1, wherein the carotenoids comprise carotene, lycopene, neurosporin, phytofluene, phytoene, xanthophyll, violaxanthin, crocetin, astaxanthin, and/or capsanthin.

17. The composition of claim 2, wherein the amino acid(s) is/are L-arginine, acid addition salts of L-arginine, glycine, L-glutamine, amides of L-glutamine, L-methionine or combinations thereof.

18. The composition of claim 3, wherein the polysaccharide(s) are amylose, pustulan, dextran, amylopectin, cellulose, arabans, fructans, glucans such as β-glucan, mannans, glycogen, chitin, pectins or combinations thereof.

19. The composition of claim 1 comprising papain, bromelain, lycopene, vitamin E acetate, vitamin A acetate and sodium selenite as active constituents.

20. The composition of claim 5 comprising papain, bromelain, lycopene, vitamin E acetate, β-carotene, grapeseed flavonoids and sodium selenite as active constituents.

21. The composition of claim 5 comprising papain, bromelain, vitamin E acetate, a rutin-containing buckwheat extract, quercetin-containing onion powder and L-arginine as active constituents.

22. The composition of claim 6 comprising papain, bromelain, β-carotene, citrus flavonoids, β-glucan and L-arginine as effective constituents.

23. The composition of claim 9 comprising trypsin or chymotrypsin or pepsin; trypsin and chymotrypsin; trypsin and chymotrypsin and pepsin; chymotrypsin and pepsin; trypsin and pepsin.

24. A food product for food supplementation comprising the composition of claim 1, wherein said food product contributes to a balanced diet, strengthens the immune defenses or the body's defenses, treats inflammatory and inflammatory-rheumatic disorders, regulates the immune system in situations of physical and/or mental stress, strengthens the immune system before, during and/or after an oncolytic treatment or reduces the risk of tumor recurrences and secondaries.

25. A medicament comprising the composition of claim 1, wherein said medicament strengthens the immune defenses or the body's defenses, treats inflammatory and inflammatory-rheumatic disorders, regulates the immune system in situations of physical and/or mental stress, strengthens the immune system before, during and/or after an oncolytic treatment or reduces the risk of tumor recurrences and secondaries.

26.-27. (canceled)

28. A dietetic treatment method comprising administering to a patient in need for such a treatment the composition of claim 1.

29. The method of claim 28, wherein said administration contributes to a balanced diet, regulates the immune system in situations of physical and/or mental stress, strengthens the immune defenses or the body's defenses, treats inflammatory and inflammatory-rheumatic disorders, strengthens the immune system before, during and/or after an oncolytic treatment or reduces the risk of tumor recurrences and secondaries.

30. A method for supplementing a persons food comprising administering to such a person the composition of claim 1.

31. The method of claim 30, wherein said administration contributes to a balanced diet, regulates the immune system in situations of physical and/or mental stress or strengthens the immune defenses or the body's defenses.
